(19) United States
(12) Patent Application Publication
(10) Pub. No.: US 2004/0102699 A1
(43) Pub. Date: May 27, 2004

(54) Tool and method to produce orientation marker on a subject of interest

(75) Inventors: Steven L. Fors, Chicago, IL (US); Mark M. Morita, Arlington Heights, IL (US); Charles Cameron Brackett, Naperville, IL (US)

Correspondence Address: GE MEDICAL SYSTEM, C/O FOLEY & LARDNER, 777 EAST WISCONSIN AVENUE, MILWAUKEE, WI, 53202-5367, US

(73) Assignee: GE Medical Systems Information Technologies, Inc.

(21) Appl. No.: 10/305,304

(22) Filed: Nov. 26, 2002

(51) Int. Cl. A61B 5/05

(52) U.S. Cl. 600/426

(57) ABSTRACT

A method and tool for producing an orientation marker on an image of a subject of interest displayed on an imaging system. The tool comprises a mechanism configured to obtain the image of the subject of interest. A means for interactively moving in real time, through the image. A means for selecting, in real time, a point on the image. A means for aligning guidelines on the selected point to define a region of the subject of interest. A means for determining, in real time, the in-plane orientation of the image relative to the defined region and the selected point, and displaying the image with the selected point and the defined region.

### BACKGROUND OF THE INVENTION

The present invention relates to medical imaging and more particularly to placing a marker on a subject of interest displayed on a computer screen to define a region of the subject of interest.

Mammograms are used as a diagnostic tool in the treatment of cancer. Computerized analysis of mammographic parenchymal patterns may provide an objective and quantitative characterization and classification of the pattern, which may be associated with breast cancer. Computerized assessment of cancer risk based on analysis of mammograms is an accepted and useful tool in treatment of cancer.

The breast is composed primarily of two components, fibroglandular tissue and fatty tissue. The average breast consists of approximately 50% fibroglandular tissue and 50% fat. Most breast carcinomas can be seen on a mammogram as a mass, a cluster of tiny calcifications or a combination of both. Other mammographic abnormalities are of lesser specificity and prevalence than masses and/or calcifications, and include skin or nipple changes, abnormalities in the axilla, asymmetric density, and architectural distension.

Studies show that use of screening mammography can reduce lesion size and stage of detection, improving the prognosis for survival. Currently, mammography is a well established imaging technique for early detection of breast cancer.

Clinical acquisition of x-ray mammograms is a rather complicated procedure and require specific techniques in order to obtain high quality images. Attenuation differences between various structures within the breast contribute to image contrast. Due to the similar composition of breast structures and the physical manifestation of breast carcinoma, screen-film mammographic imaging must be substantially different from general radiographic imaging. Low energy x-rays are required to enhance the ability to differentiate between normal tissues and carcinoma. The radiological appearance of the breast varies between individuals because of variations in the relative amount of fatty and fibroglandular tissue. Fat appears dark (i.e., high optical density) on a mammogram while fibroglandular tissue appears light (i.e., low optical density) on a mammogram. Regions of brightness associated with fibroglandular tissue are normally referred to as mammographic density.

Screening mammography typically includes two standard radiographic projections, medio-lateral oblique (MLO) and cranio-caudal (CC), that are taken of each breast (right and left) for a total of four images. The purpose of these two views is to completely image the breast and, if any lesions are present, allow localization and primary characterization.

At present, during a screening mammography, there is no current way for a system to define breast coordinates that segment the lateral and medial aspects of a cranio-caudal images or the upper and lower aspects of the medial lateral oblique images. Therefore, when a radiologist adds notations to the images, he/she must also state the relative position within the breast. Conventional techniques include the use of a cartoon or illustration of a breast which require the radiologist to translate diseased tissue locations from actual images to the cartoon representations. Another technique that is used for orientation is to attach, typically with tape, a metallic ball, typically brass, such as a bb, to the nipple of a woman's breast at the time the image acquisition is taking place. The metallic ball only serves as a landmark and does not automate the location descriptions of the breast in either the MLO or CC views. In addition, patients typically complain about embarrassment or discomfort of the bb's on their breast during the mammographic procedure.

Thus there is a need for a tool to produce an orientation marker on a subject of interest displayed on a medical imaging system. There is further need for a method to produce an orientation marker on an image of a subject of interest displayed on an imaging system.

### SUMMARY OF THE INVENTION

In accordance with the present invention, applicants provide a method and tool for producing an orientation marker on an image of a subject of interest displayed on an imaging system. The tool comprises a mechanism configured to obtain the image of the subject of interest. A means for interactively moving in real time, through the image. A means for selecting, in real time, a point on the image. A means for aligning guidelines on the selected point to define a region of the subject of interest. A means for determining, in real time, the in-plane orientation of the image relative to the defined region and the selected point, and displaying the image with the selected point and the defined region.

Another embodiment provides a method for producing an orientation marker on an image of the subject of interest displayed on an imaging system, such as a computer screen. The method comprises the steps of interactively moving, in real time, through the image. Selecting, in real time, a point on the image. Aligning guidelines on the selected point to define a region of the subject of interest. Determining in real time, the in-plane orientation of the image relative to the defined region in the selected point, and displaying the image with the selected point and the defined region.

Other principal features and advantages of the present invention will become apparent to those skilled in the art upon review of the following drawings, the detailed description in the appended claims.

### BRIEF DESCRIPTION OF THE DRAWINGS

FIG. 1 is a block diagram of an imaging system, used for mammography analysis, using the tool and method to produce an orientation marker on an image of a subject of interest displayed on a computer screen.

FIG. 2 illustrates guidelines marking a point defined by a user during an image acquisition process on the lateral and medial aspects of a cranio-caudal views of the left and right breast (subject of interest) of a patient.

FIG. 3 illustrates the guidelines marking a point on the medio-lateral oblique images of the left and right breast (subject of interest) of the patient.

Before explaining exemplary embodiments of the invention in detail, it is to be understood that the invention is not limited to its application on the details of construction and arrangement of the components as set forth in the following description or as illustrated in the drawings. The invention is capable of other embodiments or being practiced or carried out in various ways. For example, the subject of interest may be some other anatomical structure or a mechanical structure to which an orientation marker, a point on the subject of interest, is to be placed. Also, it is to be understood the phraseology and terminology employed herein is for the purpose of description and should not be regarded as limiting.

### DETAILED DESCRIPTION OF THE EXEMPLARY EMBODIMENTS

Before describing the exemplary embodiments of the tool and method for producing an orientation marker on an image, several general comments are appropriate. An orientation marker on an image can be on any subject of interest requiring such marker. For instance, it can be used in a mechanical structure or an anatomical structure. The examples illustrated in this disclosure are for a human breast, however, it should be understood that it is not limited to such anatomical structure.

During a mammography examination, the operator of the mammography equipment can utilize the present tool and method to identify the nipple of the breasts being imaged to immediately allow the system to have a point of reference for any annotations that are made on the images. The marker can be placed on the images at the time the image is being acquired or at some later time after retrieval of the image from a storage device such as a picture archiving communication system (PACS) associated with the equipment.

During the mammography image acquisition process, the x-ray technician or the radiologist can, with one click, identify the nipple on each image. Guidelines, which are a user configurable feature, can be toggled from an input device on the operator console of the system to align and mark the point on the subject of interest displayed on the computer screen or other viewing device. Upon marking the point on the structure of interest, the present tool defines the region of the subject of interest to which annotations will be indexed. This method is repeated for each image in the particular mammography sequence. The images with the defined region and selected point can then be archived in the storage device such as the PACS.

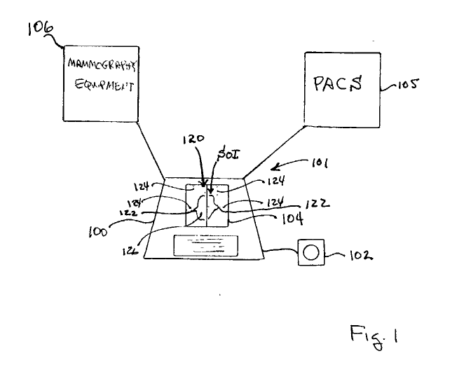

Referring now to the figures, FIG. 1 illustrates exemplary embodiments of major components of a mammography imaging system. The operation of the system is controlled from an operator console 100 which includes an input device 102, and a display screen 104. The console 100 communicates with the mammography equipment 106 and a storage device 105 that enables an operator to control the production and display of images on the screen 104. The operator console 100 can be a general purpose computer or a work station coupled to a server or mainframe which is a part of the system. It is also contemplated that the communication between the operator console 100 and the storage device 105 can be by hard wire, radio or optical transmission of energy represented of the information of interest.

The storage device 105 can include, but is not limited to, any type of disk including floppy disks, optical disks, CD ROMs and magneto-optical disks, ROMs, RAMs, EPROMs, EE PROMS, magnetic and optical cards, or any type of media suitable for storing electronic instructions and images.

If stored in any one of the above described storage media, the system includes programming for controlling both the hardware of the system and for enabling the operator console 100 to interact with a human user. Such programming may include, but is not limited to, software for implementation of device drivers, operating systems, and user applications. Such computer readable media further includes programming or software instructions to direct the system to perform the tasks in accord with the present invention.

Referring to FIG. 2, there is illustrated a cranio-caudal view of the left and right breast 116 of a patient displayed on a computer screen 104. A cranio-caudal view is a view of the subject of interest (SOI) as viewed from above the anatomical structure 115. The mechanism 106, in this case, mammography equipment, is configured to obtain the image 120 of the subject of interest (SOI). The image 120 is displayed on the computer screen 104. The user of the system interactively moves through the image 120 using the input device 102. The user selects a point 122 on the image 120 to which guidelines 124 are aligned. The guidelines 124 on the selected point 122 define a region 126 of the subject of interest (SOI). In the illustrated example, the guidelines 124 cross at right angles, at the tip of the nipple 117 of the depicted breast 116 at each of the left and right breasts 116. The guidelines 124 define two regions 126 of the subject of interest (SOI) on either side of the horizontal guideline. It should be noted that each image must have the orientation marker and point 122 defined since registration of the images is not going to be exact. With the point 122 aligned on the image 120, the tool 101 determines the in-plane orientation of the image 120 relative to the defined region 126 and the selected point 122 and displays the image 120 with the selected point 122 and the defined region 126 on the computer screen 104.

It should be noted that the use of the tool 101 and placement of the point 122 can be done in real time as the image is being acquired or it can be done on an archived image retrieved from a storage device 105 at some other point in time. It is also contemplated that edge enhancements can be implemented that would automatically detect the nipple 117 based on the geometry of the image.

The various actions associated with the present tool 101 such as the interactively moving, selecting, aligning and determining the point on the image, is typically done with the input device 102. The input device 102 can be selected from a group including a mouse, a joy stick, a keyboard, touch ball, touch screen, a voice recognition control, and a light wand. For example “clicking” on the tip of the nipple 117 instructs the system to place the guidelines 124 at the selected point 122 on the image 120. The input device 102 can also be used for changing the image being acted upon, or for reconfiguring the guidelines 124 and for instructing the system to store and retrieve the displayed image 120. The input device 102 can also be used for annotating the displayed image 120 in the defined region 126. Such annotations can be done by predefined notations, by dictation or voice recognition system, or by typing at a keyboard.

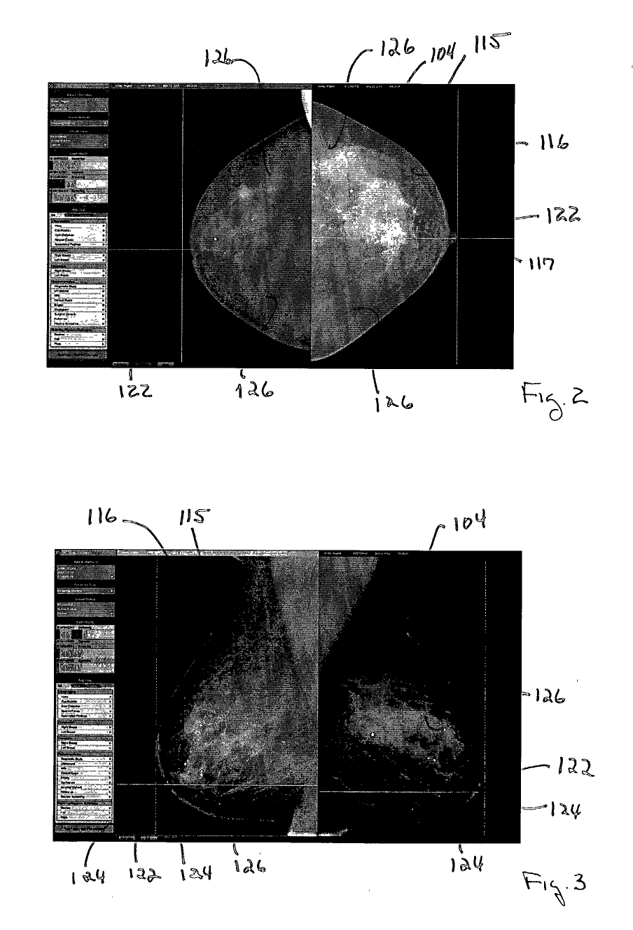

Referring to FIG. 3, there is illustrated the medio-lateral oblique views (side views) of the left and right breast 116 of an anatomical structure 115 of the subject of interest (SOI). The guidelines 124 and the point 122 which is at the tip of a nipple 117 in each view is placed on the image 120 as described above.

The user of the system, such as a radiologist or other health care professional, can select one of the images 120 from the group consisting of a lateral and medial aspects of the cradio-caudal images as illustrated in FIG. 2 or the upper and lower aspects of a medio-lateral oblique image of the subject of interest (SOI) as depicted in FIG. 3 or other types of views, such as a magnification view, a cleavage view and a spot compression (spot view). With the nipple 117 marked by the selected point 122 in each of the images, the system has a reference for any annotations created by the user when describing the tissue structure and other analysis of the images depicted on the computer screen 104.

An advantage of using this tool and method provides a one click tool that defines specific regions of the subject of interest (SOI) and eliminates the mental and verbal descriptions that a user would otherwise have to use in describing the various regions on the subject of interest (SOI). After the images are acquired, they are reviewed by the radiologist. If a mass is seen on the breast, the radiologist needs to describe that mass along with its location in the breast. The nipple marker enables not only the physician to clearly identify the location of the mass within the breast, but also provides the system with the ability to understand the regions of the breast as they are in the image. The value is that, providing the system with this information allows for additionally features to be automated by the system. For example, Structured Reporting can be overlayed in that the physician now only needs to identify and measure the mass and the system can automate the location for the report. Additionally, CAD (Computer Aided Diagnosis) can be further supplemented in that CAD can find the mass, but may not know its location. The two together will provide for a more complete automated mammography screening.

It is also contemplated that the guidelines 124 can be reconfigured by the tool 101 and method described herein from a solid line as depicted in FIGS. 2 and 3 to a broken line or some other suitable and satisfactory representation of the guidelines 124. It is also contemplated that different colors can be utilized for the guidelines 124 and the selected point 122 of interest on the subject of interest (SOI) as determined by the user.

Thus, there is provided a tool and method to produce an orientation marker on an image of a subject of interest (SOI) displayed on a computer screen. Obviously, numerous modifications and variations of the described method and tool are possible in light of the above teachings. It is therefore to be understood that within the scope of the appended claims, the invention may be practiced than as otherwise as specifically described herein.

What is claimed is:

1. An tool to produce an orientation marker on an image of a subject of interest displayed on a computer screen, the tool comprising:
a mechanism configured to obtain the image of the subject of interest;
means for interactively moving, in real-time, through the image;
means for selecting, in real-time, a point on the image;
means for aligning guidelines on the selected point to define a region of the subject of interest;
means for determining, in real-time, the in-plane orientation of the image relative to the defined region and the selected point; and
displaying the image with the selected point and the defined region.

2. The tool of claim 1, including a means for changing the image being acted upon.

3. The tool of claim 1, including a means for reconfiguring the guidelines.

4. The tool of claim 1, including a means for storing the displayed image.

5. The tool of claim 1, including means for annotating the displayed image in the defined region.

6. The tool of claim 1, wherein the displayed image in one selected from a group consisting of magnification view, cleavage view, spot view, lateral and medial aspects of a cranial caudal image and upper and lower aspects of a medial lateral oblique image of the subject of interest.

7. The tool of claim 6, wherein the subject of interest is an anatomical structure.

8. The tool of claim 7, wherein the anatomical structure is a breast.

9. The tool of claim 8, wherein the point selected is on the breast nipple.

10. The tool of claim 1, wherein the means for selecting is performed by an input device selected from a group consisting of a mouse, a joystick, a keyboard, a track ball, a touch screen, a voice recognition control and a light wand.

11. A method for producing an orientation marker on an image of a subject of interest displayed on a computer screen, the method comprising the steps of:
interactively moving, in real-time, through the image;
selecting, in real-time, a point on the image;
aligning guidelines on the selected point to define a region of the subject of interest;
determining, in real-time, the in-plane orientation of the image relative to the defined region and the selected point; and
displaying the image with the selected point and the defined region.

12. The method of claim 11, including a step of changing the image being acted upon.

13. The method of claim 11, including a step of reconfiguring the guidelines.

14. The method of claim 11, including a step of storing the displayed image.

15. The method of claim 11, including a step of annotating the displayed image in the defined region.

16. The method of claim 11, wherein the displayed image in one selected from a group consisting of magnification view, cleavage view, spot view, lateral and medial aspects of a cranial caudal image and upper and lower aspects of a medial lateral oblique image of the subject of interest.

17. The method of claim 16, wherein the subject of interest is an anatomical structure.

18. The method of claim 17, wherein the anatomical structure is a breast.

19. The method of claim 18, wherein the point selected is on the breast nipple.

20. The method of claim 11, wherein the step of selecting is performed by an input device selected from a group consisting of a mouse, a joystick, a keyboard, a track ball, a touch screen, a voice recognition control and a light wand.
